(19) United States
(12) Patent Application Publication
(10) Pub. No.: US 2013/0231551 A1
(43) Pub. Date: Sep. 5, 2013

(54) SYSTEM AND METHOD FOR CARDIOVASCULAR EXERCISE STRESS MRI

(71) Applicants: Orlando Paul Simonetti, Columbus, OH (US); Eric Lee Foster, Columbus, OH (US); John Wayne Arnold, New Philadelphia, OH (US); Subha V. Raman, Columbus, OH (US)

(72) Inventors: Orlando Paul Simonetti, Columbus, OH (US); Eric Lee Foster, Columbus, OH (US); John Wayne Arnold, New Philadelphia, OH (US); Subha V. Raman, Columbus, OH (US)

(73) Assignee: THE OHIO STATE UNIVERSITY, Columbus, OH (US)

(21) Appl. No.: 13/601,483

(22) Filed: Aug. 31, 2012

Related U.S. Application Data
(63) Continuation of application No. 12/424,835, filed on Apr. 16, 2009, now abandoned.
Application No. 12/424,835 is a continuation of application No. PCT/US07/81948, filed on Oct. 19, 2007.
(60) Provisional application No. 60/862,107, filed on Oct. 19, 2006.

(51) Int. Cl. A61B 5/00; A61B 5/02

(52) U.S. Cl. CPC A61B 5/0044; A61B 5/02
USPC 600/410; 482/8

(57) ABSTRACT

A system and method for cardiovascular exercise stress magnetic resonance using a MR-compatible treadmill. The treadmill is made of non-ferromagnetic materials and has a belt driven by a motor that produces rotational motion from pressurized fluid that is produced by a pump located outside the scan room. The treadmill is adjacent to the MR imager. Patients complete an exercise protocol on the treadmill and are then transferred to the MR imager without leaving the scan room. Images are acquired as quickly as possible post-exercise to more accurately diagnose cardiovascular disease in patients.

### CROSS-REFERENCE TO RELATED APPLICATIONS

This application is a continuation under 35 USC §120 of application Ser. No. 12/424,835 filed Apr. 16, 2009, which is a continuation of application number PCT/US07/81948, filed Oct. 19, 2007, titled SYSTEM AND METHOD FOR CARDIOVASCULAR EXERCISE STRESS MRI, which is in turn entitled to benefit of a right of priority under 35 USC §119 from U.S. Ser. No. 60/862,107, filed Oct. 19, 2006, titled MAGNETIC RESONANCE COMPATIBLE TREADMILL, both of which are herein incorporated by reference in their entirety.

### BACKGROUND OF THE INVENTION

The invention relates to cardiac stress testing, and more particularly relates to cardiac stress testing using diagnostic medical imaging techniques. In its most immediate sense, the invention relates to cardiac exercise stress testing using magnetic resonance (“MR”) imaging.

In an electrocardiogram (ECG) cardiac exercise stress test, a patient is connected to an ECG monitor and instructed to walk on a treadmill following an exercise protocol such as the Bruce treadmill protocol. As the test continues, the speed and inclination of the treadmill are gradually increased until the patient reaches peak effort (thereby generating peak cardiovascular stress). The test stops when the patient reaches his or her maximum heart rate, an ECG abnormality is detected, or when the patient cannot continue the exercise, whichever occurs first. The results of the test are then used to assess the patient's heart function.

Cardiac exercise stress tests are also carried out using medical diagnostic imaging techniques. Such techniques typically require the acquisition and comparison with each other of two images of the heart; a pre-exercise image acquired when the patient's heart is at rest is compared with a post-exercise image acquired after the patient has exercised.

For example, when nuclear medicine is used as the imaging modality, a first dose of a radioisotope is introduced into the patient's bloodstream while the patient is at rest. The blood circulates and perfuses the patient's myocardium, and the radioisotope is taken up in the resting myocardium. The patient's heart is then imaged in a gamma camera, which responds to the radioisotope to form a pre-exercise image showing myocardial perfusion while the patient is at rest. Then, the patient is exercised on a treadmill, and instructed to tell the clinician when (s)he is about to reach maximum effort. A second dose of radioisotope is then administered, and the patient continues to exercise for a period (perhaps 90 seconds) during which the blood perfuses the patient's myocardium and the radioisotope is taken up in the patient's myocardium while the heart is under stress. Several minutes later (perhaps 30 minutes later) the patient's heart is then once again imaged in the gamma camera to form a post-exercise image showing post-exercise myocardial perfusion. (Significantly, a nuclear medicine cardiac exercise stress test does not require that the patient's heart be imaged while the patient's heart is at peak stress. This is because the nuclear medicine image detects the distribution of the radioisotope within the myocardium and this distribution does not change rapidly.)

Echocardiography can also be employed as the imaging technique. In a cardiac exercise stress test using echocardiography, the two sets of heart images are acquired using an ultrasound imager, with the pre-exercise images of the heart being compared with the post-exercise images of the patient's heart acquired immediately following the time the patient has reached peak effort by exercising on a treadmill.

An MR imager can produce high-resolution images quickly and for these reasons researchers have tried various approaches for using MR imaging in cardiac stress testing. All have been unsatisfactory. One approach was to induce stress pharmacologically rather than by exercise on a treadmill. This was unsatisfactory because pharmacologically induced stress does not link physical activity to symptoms as treadmill exercise does. Another approach required the patient to exercise on a supine bicycle ergometer located inside the imager. While this was useful for certain research studies, it was unsuitable for cardiac stress testing. This is because the diagnostic value of the post-exercise MR image is maximized when that image is acquired at peak effort. Patients were unable to reach peak effort by cycling in a supine position because it was uncomfortable and because leg fatigue caused patients to cease exercising before they reached peak effort. In still another approach by Rerkpattanapipat et al., a treadmill was placed outside the scan room in which the imager was located. (The necessity for placing the treadmill outside the scan room is discussed in more detail below.) Patients exercised on the treadmill and then walked twenty feet to the imager in order to acquire the post-exercise heart image. Although the results of this experiment were encouraging, the Rerkpattanapipat et al. experimental setup is not practical for clinical use. This is because a patient's heart rate slows down between the time that exercise ceases and the time that the patient is imaged in the MR imager, whereby the post-exercise image of the heart is not an image of the heart at peak effort. Additionally, use of a treadmill outside the scan room raises safety concerns with moving a patient such a distance after (s)he has reached peak effort.

As stated, in the Rerkpattanapipat et al. experiments, the treadmill was located outside the scan room in which the MR imager was located. This was necessary because it would have been unwise and indeed unsafe to place a conventional treadmill in the scan room. An MR imager necessarily generates an intense magnetic field and a conventional treadmill contains ferromagnetic components (particularly the electric motor used to drive the treadmill belt). If a ferromagnetic object is in the scan room, the object will likely be pulled towards the imager with great force, potentially causing great damage to the imager and even causing serious injury to the patient. For example, in one reported instance an oxygen tank located in the scan room was turned into a projectile when the imager was energized, causing injury to a pediatric patient. Additionally, even if a ferromagnetic component were to be firmly fixed in position so that it could not come loose to cause equipment damage or patient injury, its presence in the scan room would be highly undesirable because it would distort the main magnetic field created by the imager. This distortion would generate artifacts in the reconstructed MR image and possibly reduce the diagnostic utility of that image. For these reasons, Rerkpattanapipat et al.'s treadmill was located outside the scan room.

It would be advantageous to provide method and apparatus for conducting a cardiac exercise stress test using MR imaging, the method and apparatus permitting a patient to exercise on a treadmill located inside the scan room immediately adjacent the MR imager.

It is therefore one object of the invention to provide method and apparatus for conducting a cardiac exercise stress test using MR imaging, which permit a patient to exercise on a treadmill located inside the scan room immediately adjacent the MR imager.

Another object is, in general, to improve on known method and apparatus of this general type.

### SUMMARY OF THE INVENTION

The invention proceeds from the realization that an electric motor is the most massive ferromagnetic element in a conventional treadmill and that replacing that element with a non-ferromagnetic motor can greatly simplify the task of providing a treadmill that can be used inside the scan room. The invention further proceeds from the realization that if the treadmill motor that drives the belt is designed to produce rotational motion from pressurized fluid supplied to the motor, the motor can be made of non-ferromagnetic components and the fluid can be pressurized by a pump located outside the scan room.

In apparatus in accordance with the invention, a treadmill is provided. The treadmill is located in the scan room and has a belt driven by a motor that produces rotational motion from pressurized fluid supplied to the motor. A pump is further provided. The pump is located outside the scan room, produces pressurized fluid, and is operatively connected to the motor. Advantageously, and in the preferred embodiment, the fluid is water-based, and the treadmill is entirely made of non-ferromagnetic materials.

Apparatus in accordance with the invention can be safely put inside the scan room because there is little (advantageously, no) ferromagnetic material to be attracted to the magnet or to distort the main field of the MR imager. Any appreciable quantity of ferromagnetic material is relocated out of the scan room where it cannot be attracted to the magnet of the MR imager and cannot adversely affect the quality of the MR image.

In a method in accordance with the invention, a treadmill is located inside the scan room adjacent the MR imager. The treadmill has a motor that drives the belt. The motor produces rotational motion in response to pressurized fluid supplied to the motor. Fluid is pressurized outside the scan room and the patient is caused to stand on the belt. The pressurized fluid is supplied to the motor to drive the belt, thereby exercising the patient inside the scan room. Then, the supply of pressurized fluid to the treadmill motor is terminated, whereby the patient's exercise ceases. The patient is then moved from the treadmill belt into the MR imager without leaving the scan room, and the MR imager is used to conduct an imaging study on the patient.

By using a method in accordance with the invention, the patient is very close to the MR imager and there is only a short time lapse between cessation of the patient's exercise and acquisition of the post-exercise MR image. It is therefore possible to acquire an MR image that is closely representative of an image of the patient's heart at peak effort.

### BRIEF DESCRIPTION OF THE DRAWINGS

FIG. 1 is an illustration of components and the configuration of components for apparatus according to an example embodiment of the present invention.

FIG. 2 is an equipment layout and a schematic diagram of an electric motor driven pump for powering a treadmill mounted hydraulic motor through hoses according to an example embodiment of the present invention.

FIG. 3 is a diagram of a non-ferromagnetic stainless steel hydraulic motor for apparatus according to an example embodiment of the present invention.

FIG. 4 is a diagram of a hydraulic drive and elevation system for apparatus according to an example embodiment of the present invention.

FIG. 5 is a schematic diagram of hydraulic components for apparatus according to an example embodiment of the present invention.

FIG. 6 is an exemplary display of a computer for a treadmill control system according to an example embodiment of the present invention.

FIG. 7 is a configuration diagram for patient positioning equipment according to an example embodiment of the present invention.

FIG. 8 is an exercise CMR protocol according to an example embodiment of the present invention.

### DETAILED DESCRIPTION OF EXAMPLE EMBODIMENTS

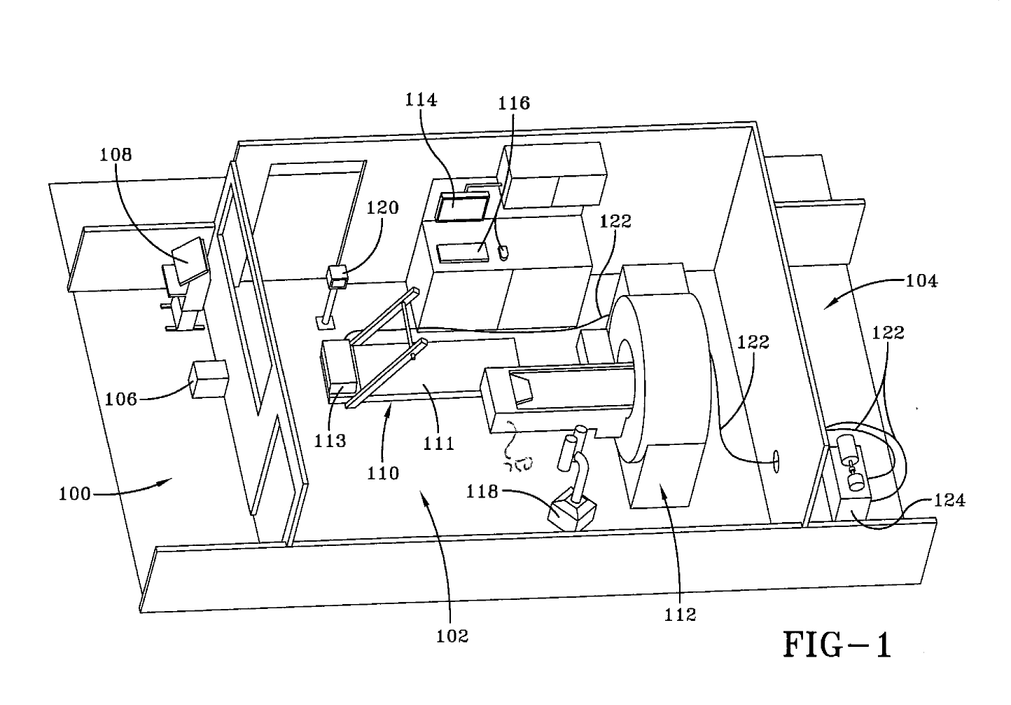

Referring to FIG. 1, an illustration of components and the configuration of components for a MR-compatible treadmill system according to an example embodiment of the present invention is shown. In this example, components of the present invention are contained in a control room 100, a scan room 102 and an equipment room 104. One or more computers 106 in the control room 100 support control and monitoring of components in the scan room 102. A treadmill control system computer may be used to communicate with the treadmill 110. Another scanner computer may be used to communicate with the MR imager 112. The scanner computer may be used to control functionaltiy of the MR imager 112 related to data acquisiton, image reconstruction, and image display and analysis.

A hydraulic powered treadmill 110 and MR imager 112 are contained in the scan room 102. The hydraulic powered treadmill 110 is connected to a hydraulic power pack 124 via hydraulic hoses 122. In FIG. 1, certain hose segments are shown as including only one hose for clarity purposes. Thus, it should be recognized that there may be more than one hose in any of the hose segments as needed. The hydraulic power pack 124, which may contain ferromagnetic components, is contained in a separate equipment room 104. In other exemplary embodiments, a hydraulic power pack may be located in any suitable location including, but not limited to a room, such that the hydraulic power pack is not adversely affected by a MR imager. A wireless monitor 114 and wireless keyboard/mouse 116 may be used in the scan room 102 to control the components housed in the scan room 102. In other exemplary embodiments, a monitor and keyboard/mouse, each of which may or may not be wireless, may be situated in any other suitable location, including outside the scan room 102, to allow effective control of any desired components.

Because as discussed below the treadmill 110 is advantageously fully MR compatible, it may be placed immediately adjacent to the MR imager 112 or in any desired position within the scan room 102. Complete MR compatibility also allows the treadmill 110 to be used adjacent to MR imagers operating at high field strengths, e.g., 3.0 Tesla. The resulting configuration of the scan room 102 may be very similar to the setup for a standard exercise echocardiography lab.

An exemplary embodiment of a treadmill 110 may comprise a support and a belt 111 rotatably mounted within the support. Furthermore, the treadmill 110 may include programmable components so as to be controlled independently through a standard PC as well as with leading treadmill stress testing software. The programmable components may execute any of a variety of exercise protocols, including the standard Bruce Treadmill Exercise protocol. The Bruce Treadmill Exercise protocol automatically advances a patient through set stages of speed and elevation as shown in Table 1. The treadmill may also or alternatively be controlled manually.

TABLE 1
Standard Bruce Exercise Protocol for Stress Testing

| Stage | Speed (mph) | Grade (%) |
|---|---|---|
| 1 | 1.7 | 10 |
| 2 | 2.5 | 12 |
| 3 | 3.4 | 14 |
| 4 | 4.2 | 16 |
| 5 | 5.0 | 18 |
| 6 | 5.5 | 20 |
| 7 | 6.0 | 22 |

Traditional treadmills include electric motors having ferromagnetic components with significant mass. As explained above, ferromagnetic items can pose a severe hazard to MR imagers and patients if they are close to an operating MR imager. An electric motor must contain ferromagnetic parts. For this reason, in an example embodiment of the present invention, a hydraulic motor 113 made of stainless steel (which is not ferromagnetic) is used to power the treadmill 110. The other components in the treadmill 110 may be made from non-ferromagnetic materials including, but not limited to, stainless steel or aluminum such that the treadmill 110 may be safely operated in close proximity to the MR imager 112 while the MR imager 112 is operating. Material choices may depend on tradeoffs between the necessary strength of the material compared to the increased cost of using stainless steel, for example.

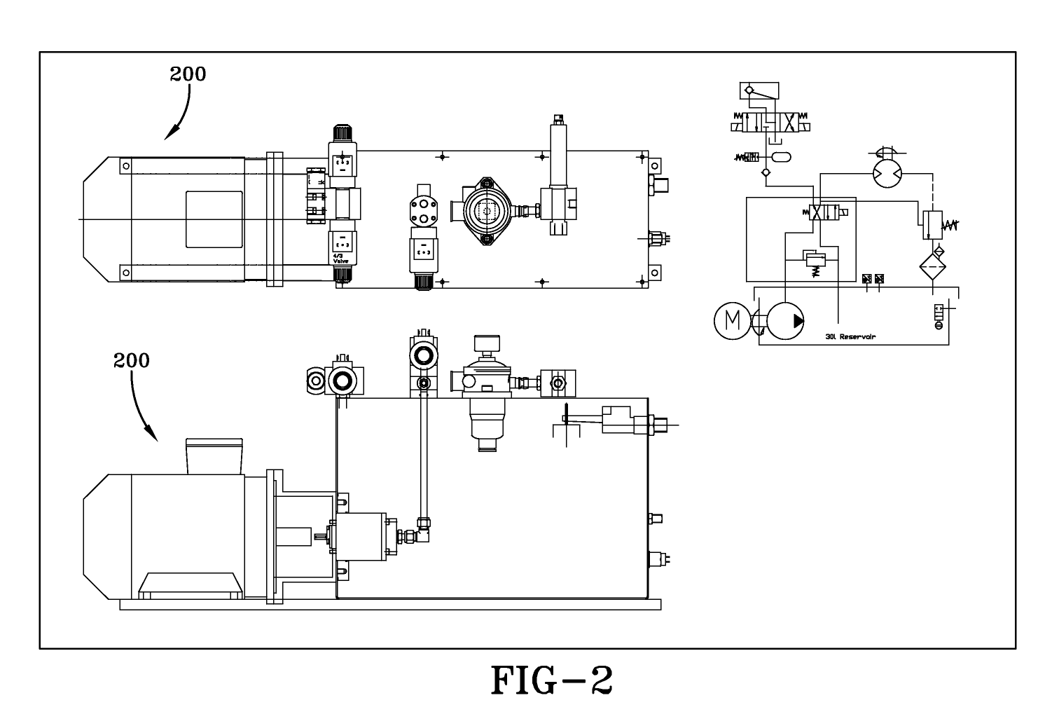

In this example, hydraulic power pack 124, comprising an electrical motor driven pump 200 as shown in FIG. 2, is located in an equipment room 104 outside the scan room 102. The pump 200 forces hydraulic fluid from a reservoir into hydraulic hoses 122 that carry the pressurized fluid into the scan room 102 to the hydraulic motor 113, which may be mounted on the front (or other suitable location) of the treadmill 110.

In an MR imaging study in accordance with a method in accordance with the invention, a patient completes all or part of an exercise protocol on the treadmill 110 and then takes his or her place within the MR imager 112. Medical staff, who may be present in the room during the stress test, may assist the patient in transferring from the treadmill 110 to the MR imager 112. The lift system of the treadmill 110 may be used to assist in transferring the patient by positioning the height of the treadmill 110 to allow easy transfer of the patient to the patient table 750 of the MR imager 112. Because the treadmill 110 may be directly adjacent to the MR imager 112, some patients may not require assistance while transferring from the treadmill 110 to the MR imager 112. Cardiac image data is acquired by the MR imager 112 and analyzed by a medical professional following patient exercise.

FIG. 1 further shows patient monitoring equipment according to an example embodiment of the present invention. Continuous 12-Lead ECG monitoring of the patient may be used during exercise. A standard 12-lead ECG system 108 may be used by positioning it at the entrance to the scan room 102, close enough to monitor the patient both on the treadmill 110 and on the patient table 750 of the MR imager 112 when the patient is outside of the magnet bore. Alternatively, the ECG system 108 may also be positioned in the adjacent control room 100 (as shown), with cabling run through the wall into the scan room 102 to the patient on the treadmill 110. While inside the bore of the MR imager 112, the ECG is non-diagnostic due to magneto-hydrodynamic artifacts caused by blood flow within the magnetic field. However, heart rate and rhythm may be monitored continuously with a wireless ECG unit. In an example embodiment of the present invention, such a wireless ECG unit may be provided by MRI manufacturer Siemens Medical Solutions, Malvern, Pa. Other MR-compatible wireless ECG systems are commercially available. This setup allows medical staff to quickly disconnect the patient from the 12-lead ECG system after exercise, while continuing to monitor heart rate. MR-compatible manual and automatic non-invasive blood pressure equipment 120 such as that from Medrad, Inc., Pittsburgh, Pa. may be used to monitor blood pressure before, during, and after the stress test.

The MR imager 112 is controlled via in-room wireless monitor 114 and wireless keyboard/mouse 116 such as are available from Siemens Medical Solutions, Malvern, Pa., and a start button located on the front panel of the MR imager 112. The wireless monitor 114 and wireless keyboard/mouse 116, designed primarily for interventional MRI applications, duplicates the functionality of the computer 106. A power contrast injector 118 such as one from Medrad Corp., Pittsburgh, Pa. may be outfitted with a manual control switch for operation from within the MRI scan room 102. The injection protocol may be pre-programmed and loaded so that it can be executed immediately at the start of the perfusion scan from within the scan room 102. In an example embodiment of the present invention, all equipment necessary to conduct the treadmill exercise test with continuous ECG 108 and blood pressure monitoring 120, as well as the equipment necessary to control the MR study, is positioned to allow the test to be performed within the scan room 102. As a result, the stress testing team is able to remain in the room and in direct communication with the patient at all times.

FIG. 2 is an equipment layout and a schematic diagram of an electric motor driven pump 200 for powering a treadmill mounted hydraulic motor through hoses according to an example embodiment of the present invention. One example of an electric motor driven pump 200 is commercially available from The Water Hydraulics Co. Ltd.

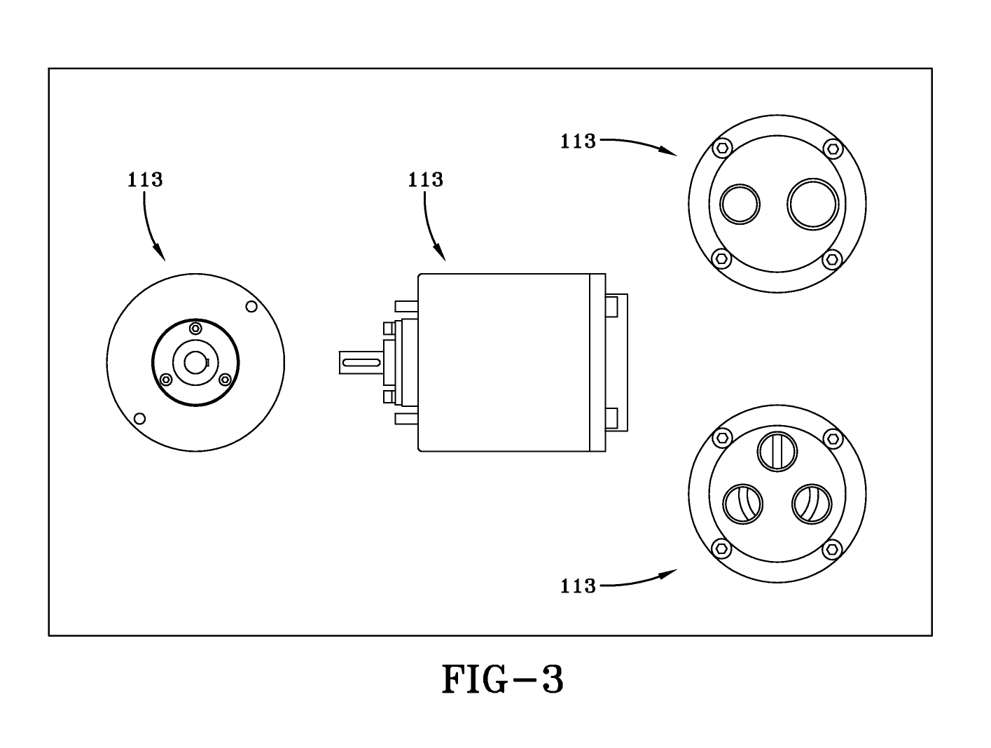

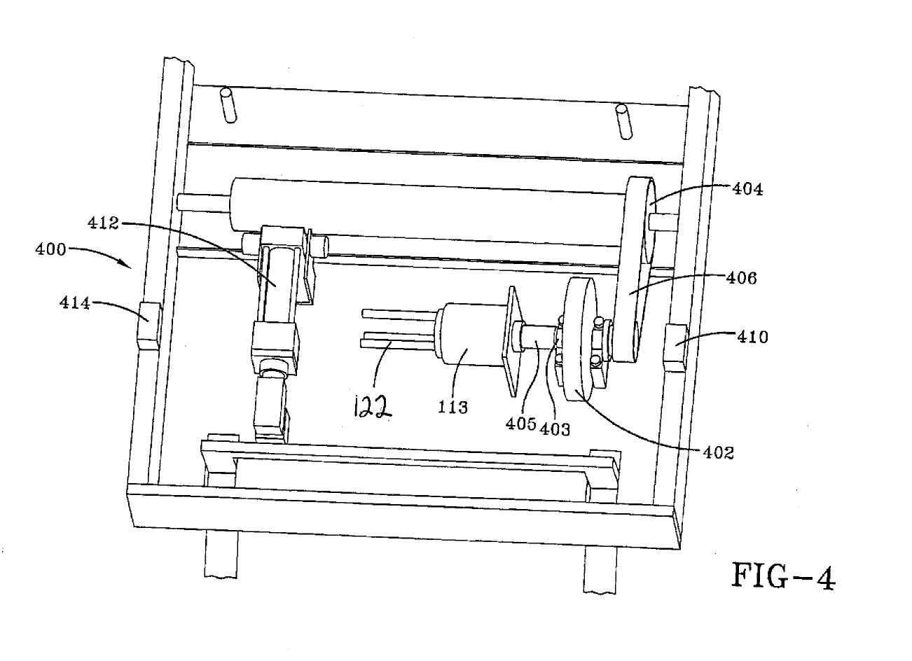

In this example, hydraulic fluid flowing from power pack 124 through the hoses 122 powers the hydraulic motor 113 (FIG. 3). The hydraulic motor 113, which in the preferred embodiment is stainless-steel, is commercially available from The Water Hydraulics Co. Ltd. Hydraulic motor 113 operates a hydraulic drive within a hydraulic drive and elevation system 400 (FIG. 4) (shown without a cover for clarity). The hydraulic drive and elevation system 400 is made of non-ferromagnetic materials such as stainless steel. In this example, a non-ferromagnetic flywheel 402 attached to a driveshaft 403 (via shaft coupler 405) attenuates inertial differences during footplant and speed change. The flywheel 402 is connected to a drive roller 404 by a drive belt 406. Drive roller 404 supports the treadmill belt 111, whereby the drive roller 404 rotates the treadmill belt 111. There may be more than one drive roller 404 to facilitate desired rotation of treadmill belt 111. Hoses 122 include at least one return hose and at least one input hose. The return hose cycles the hydraulic fluid back to the reservoir. In this exemplary embodiment, hoses 122 are attached to the treadmill 110 via MR-compatible, hydraulic quick couplings to allow for quick, clean setup and teardown.

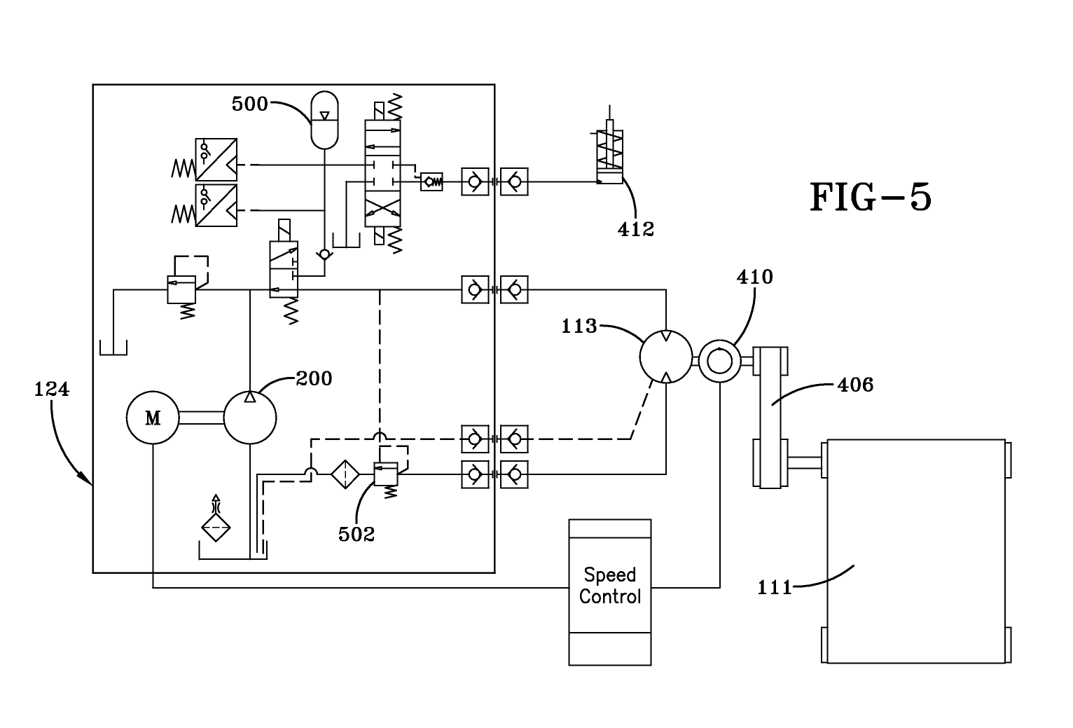

An example of the operation of one embodiment will now be described. A variable speed electric motor in the power pack 124 drives the hydraulic pump 200 (FIG. 2) in the power pack 124 via a shaft and flexible coupler. The hydraulic pump 200 pressurizes the hydraulic fluid which is supplied to the hydraulic motor 113 by the hoses 122. The flow rate of the hydraulic fluid is proportional to the rotational speed of the pump and the pressure of the hydraulic fluid is proportional to the load on the treadmill 112. The fixed displacement hydraulic motor 113, which is mechanically coupled to the treadmill belt 111 via drive shaft 403, drive belt 406, and drive roller 404, produces rotational motion in response to the pressurized fluid that is supplied to it. In the ideal case, the speed of the hydraulic motor 113 can be proportional to the speed of the electric motor, but in this example, the speed relationship only approximates direct proportionality. (This relationship is non-linear since internal leakage rates in the pump 200 and motor 113 are dependent on load, temperature and other factors.) Because of this, a feedback control system is provided to maintain speed of the treadmill belt 111 independently of load and other factors. This feedback control system includes an optical sensor 410 mounted adjacent the flywheel 402, but other suitable speed sensors may be employed instead. As shown in FIG. 5, the output from the sensor 410 is used to control the speed of the electric motor that drives the pump 200. After flowing through the hydraulic motor 113, hydraulic fluid is routed through a hydraulic braking valve 502 in the power pack 124. The valve 502 maintains appropriate fluid pressure at the outlet of the motor 113 to control the speed of the treadmill belt 111 and to prevent fluid cavitation at the inlet of the motor 113 when the treadmill belt 111 is operated at high gradients.

In further description of one example of an operation of one embodiment, the Bruce Treadmill Exercise protocol requires the treadmill to attain a maximum grade of 22% to accommodate patients with a wide range of physical conditions. The grade (gradient) of the treadmill belt 111 is controlled by an ancillary circuit located in the power pack 124. An accumulator 500 of power pack 124 is charged with a volume of fluid sufficient to operate the treadmill 110 for a complete patient test session. At each protocol stage, a portion of the stored fluid is directed to the non-ferromagnetic treadmill lift cylinder 412 by way of valves and conductors (hoses in communication with cylinder 412 are not shown in FIG. 4 for clarity purposes). As one example of an alternative, a conventional linear actuator may be positioned on the hydraulic power pack outside of the MRI room. The standard hydraulic cylinder is actuated by the linear motor and its movement replicated by the MR-compatible cylinder via hydrostatic transmission. Elevation feedback may be provided using either a linear position sensor mounted on the cylinder, or a fluid-filled tilt sensor 414 mounted to the frame of the treadmill 110 (FIG. 4, Table 2).

The remaining design elements of an exemplary system include components suitable for a safe, reliable machine. With particular reference to this example, a pressure relief valve is installed at the pump outlet. Non-ferromagnetic hoses and couplers comprise the required fluid conductors. The couplers are sized and polarized to prevent incorrect connection during equipment setup. System cooling is provided by the reservoir. Filtration is built into the power pack 124 to filter the fluid returning from the circuits as well as fluid added to the system. Level sensors and pressure switches are used to complete the control circuits.

Referring to FIG. 5, a schematic diagram of hydraulic components for a MR-compatible treadmill according to an example embodiment of the present invention is shown. Due to the placement of the treadmill system within a healthcare facility, the hydraulic power system may be designed to use water rather than traditional oil-based hydraulic fluids. Water based hydraulic fluids allow for simple cleanup of any accidental fluid leakage from the system as well as eliminate the danger of combustion of the hydraulic fluid. It also makes the system more universal by eliminating the need for on-hand stock of hydraulic fluid. Furthermore, with reference to FIG. 5, power pack 124 may include a braking valve 502 to help control belt speed.

In this example, the treadmill control system is located in the control room 100 outside the scan room 102 as shown in FIG. 1. An application executing on a control computer 106 communicates with the programmable components of the treadmill 110 to control the speed and grade of the treadmill 110. The control program of computer 106 flexibly and automatically runs the treadmill speed and elevation through a preset exercise protocol such as the Bruce Treadmill Exercise protocol or any other exercise stress protocol. The control program allows for feedback control to ensure the protocol is being followed precisely. As stated above, an optical sensor 410 positioned adjacent the flywheel 402 monitors the speed of the treadmill 110 and sends a signal back to the controller, and an angle sensor 414 mounted on the support of the treadmill 110 provides elevation feedback. For safety purposes, a manual control emergency stop button may be located on the treadmill. Additional sensors shown in Table 2 may further provide for safe operation of the hydraulic treadmill system.

TABLE 2
MR Compatible Treadmill Control System Sensor Options

| SENSORS | TYPE | LOCATION |
|---|---|---|
| Functional | | |
| Belt speed | Optical sensor with signal | Located on treadmill at |
| | carried on fiber optic cable | flywheel |
| Elevation | Option 1: | On elevation cylinder |
| | Linear potentiometer | |
| | Option 2: | Any flat surface under |
| | Fluid filled tilt sensor | hood of treadmill |
| Motor pressure | Pilot operated brake valve | Motor outlet with sensing |
| control | | from motor inlet |
| Safety | | |
| Emergency Stop | Button switch | Handle of treadmill |
| Water level | Level | Reservoir |
| Temperature | Temperature switch | Reservoir |

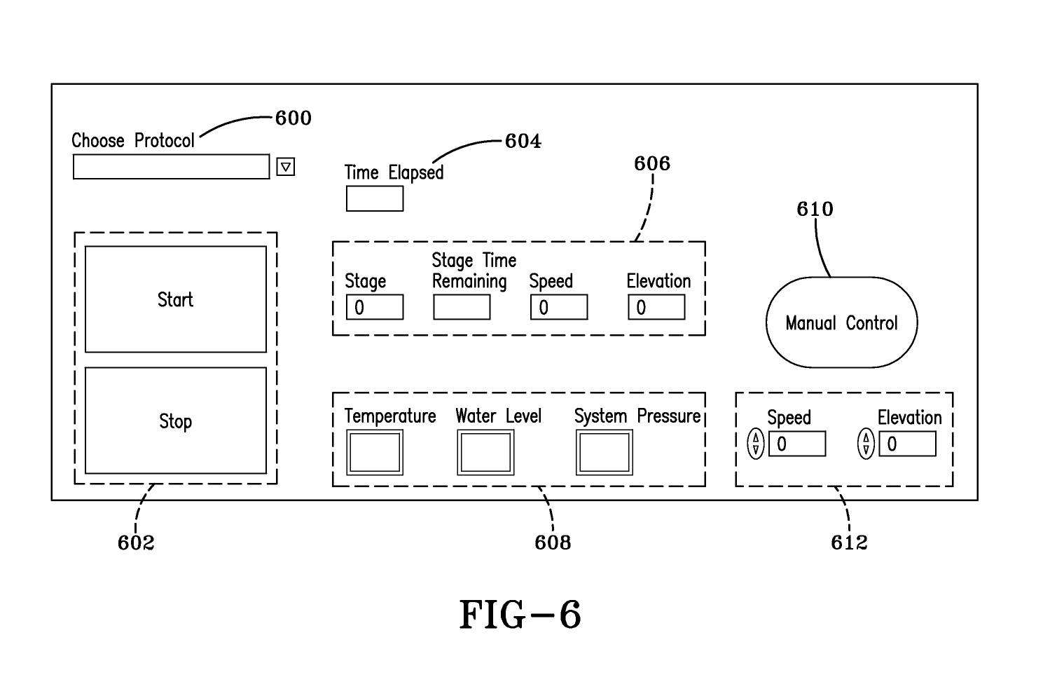

Referring to FIG. 6, a screen shot of a display for a treadmill control system according to an example embodiment of the present invention is shown. To control the operation of the treadmill both manually 610 and through a prescribed exercise protocol 600, the hydraulic motor speed and treadmill elevation 612 are input from a computer screen. Start and stop options 602 are used to start and end a selected protocol 600. The screen also shows the time elapsed 604, current stage 606 as well as time remaining in the stage and the speed and elevation for the stage. Status information 608 related to temperature, water level, and system pressure for the hydraulic system is also communicated on the screen.

A speed signal is routed to a motor controller that controls the speed of the electric motor located in the power pack 124 outside the MRI exam room. The electric motor controls the speed of a pump 200, which in turn controls the rate of fluid flow delivered to the hydraulic motor 113 located in the treadmill 110. The signal from the motor shaft speed sensor 410 is fed into a feedback loop where it is compared with the intended speed of the hydraulic motor 113. A signal is sent to the electric motor control, which alters the speed of the electric motor in the power pack 124.

At higher elevations of the treadmill belt 111, depending mainly on the weight of the patient, the work of the patient running on the treadmill 110 acts to drive the motor 113 to a speed that is higher than desired. At this point, the valve 502 is activated, creating back pressure on the hydraulic motor 113. The hydraulic motor 113 then acts as a brake, enabling the system to maintain the prescribed speed. An emergency stop button located on the body of the treadmill 110 provides a motor shutoff signal if needed.

The treadmill elevation signal is output to the elevation mechanism. The mechanism may be a pre-charged accumulator that outputs the desired quantity of fluid through a valve either to a non-ferromagnetic hydraulic cylinder or to a master-slave cylinder system in which a traditional hydraulic cylinder located outside the room controls a slave cylinder located on the treadmill. A feedback signal is received from the elevation sensor, which may be either from a linear potentiometer located on the elevation cylinder or a fluid filled tilt sensor located on any flat surface of the treadmill. This signal enters a separate feedback loop where it is compared to the intended elevation.

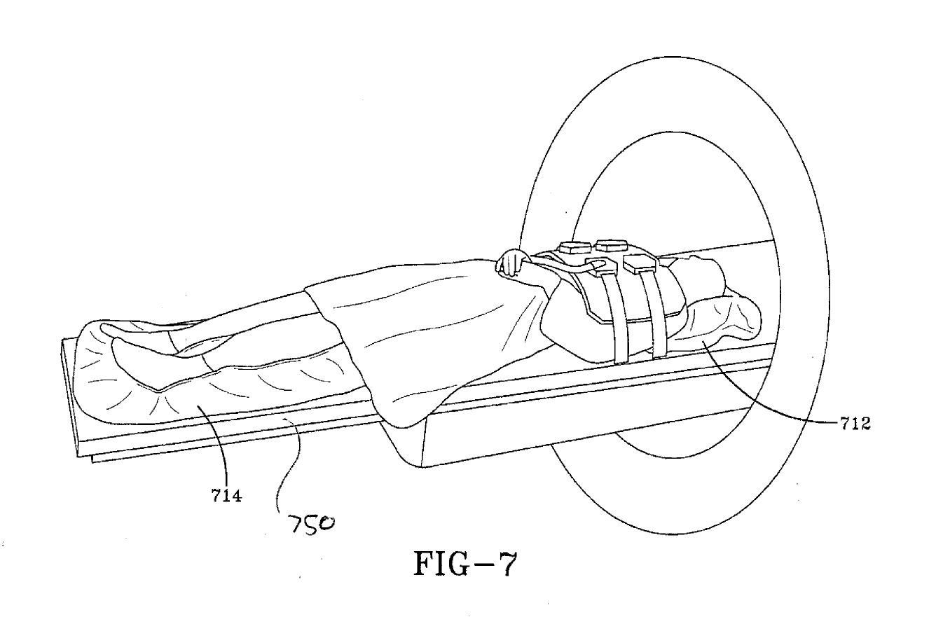

Referring to FIG. 7, a configuration diagram for patient positioning equipment according to an example embodiment of the present invention is shown. Before exercise, a patient is positioned on the patient table 750 of the MR imager 112 using two vacuum mattresses 712, 714 such as those available from Vac-Lok Cushions, MEDTEC, Orange City, Iowa, and slice localization and resting function scans are performed. One vacuum mattress is placed under the head and shoulders 712 and the other under the legs extending from foot to upper thigh 714. Removal of air with a vacuum pump causes the mattresses to rigidly conform to the body. These devices are commonly used for repositioning of patients undergoing repeated radiation therapy sessions. This system ensures that the patient returns to the same position after exercise such that stress imaging may be performed using the slice planes previously prescribed at rest.

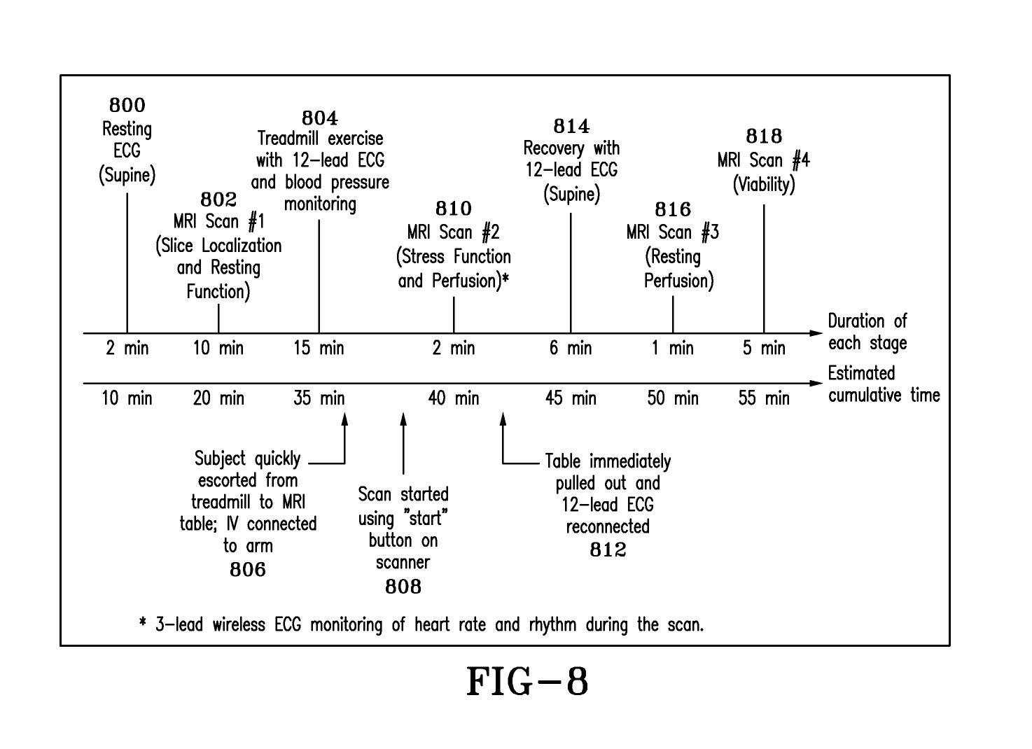

Referring to FIG. 8, an exercise CMR protocol according to an example embodiment of the present invention is shown. Patient preparation includes insertion of an intravenous (IV) needle and the standard placement of both the 12-lead and the wireless ECG electrodes on the chest. Supine 12-lead ECG and blood pressure (BP) are recorded at rest step 800. The supine resting ECG is used for direct comparison with the supine recovery ECG post-exercise. Next, patients are positioned on the patient table 750 using the vacuum mattresses 712, 714. Air is removed from the mattresses 712, 714 through a vacuum line located inside the scan room 102.

In an example embodiment of the present invention, slice localization by single-shot steady-state free precession (SSFP) imaging is followed by resting cine imaging in step 802. The cine function sequence is configured to scan each slice position for approximately 2 seconds, while the temporal resolution varies depending on the size of the patient and the resulting field of view. A test acquisition for first-pass perfusion may be performed without contrast agent. Pulse sequences are queued for stress imaging such that they may be executed automatically from the scan start button located on the MR imager 112. The patient is then removed from the MR imager 112. Certain makes and models of MR imagers may require medical staff to use extra care when removing the patient so as not to pull the patient table 750 all the way out of the MR imager 112, and not to move the surface array coil too drastically. Either of these actions may cause certain systems to repeat adjustments prior to the start of the stress scan, causing delays.

Next, the patient exercises on the treadmill 110 positioned inside the scan room 102. In an example test, the speed and elevation of the treadmill 110 are progressively increased every three minutes following the standard Bruce protocol. 12-lead ECG is continuously monitored during exercise. Blood pressure is measured and a hard copy of the ECG is obtained at the midpoint of each Bruce protocol stage. As with conventional stress testing, patients are continuously monitored by a nurse and/or physician who may stop the test at any time based on recognition of adverse endpoints or in response to the patient's request.

After reaching his or her exercise limit or the maximum predicted heart rate (MPHR) based on age (220-age), the patient is moved onto the patient table 750 of the MR imager 112 (step 806). The surface coil is placed on the chest, the contrast injector 118 is connected to a previously inserted IV in the patient's arm, and the patient table 750 is withdrawn to move the patient inside the MR imager 112. The previously prepared cine and first-pass perfusion scans are started using the start button located on the MR imager 112 (step 808); stress function is executed first, followed by stress perfusion in step 810. The time from end of exercise to start of imaging (Tstart) is recorded. A member of the medical team starts the injection protocol as soon as an audible change from the cine pulse sequence to the first-pass pulse sequence is detected. The patient remains inside the MR imager 112 for approximately 90 seconds for stress imaging.

Following exercise on the treadmill 110, MR studies are executed to evaluate cardiac function and myocardial perfusion at peak stress. In an example embodiment of the present invention, cardiac function is evaluated using a real-time steady-state free precession (SSFP) pulse sequence with TR/TE of 2.3/1.0 msec and Temporal Sensitivity Encoding (TSENSE) acceleration factor of 3. Five slices are acquired in the short axis (SAX) direction, and one slice each in horizontal (HLA) and vertical (VLA) long axis directions. Temporal resolution of 57 msec and spatial resolution of 3.0 mm×3.8 mm×8 mm may be achieved with no breath-hold and no ECG gating. Each slice position is scanned for approximately two seconds depicting three or more cardiac cycles, depending on heart-rate. Thus, cine images depicting three or more cardiac cycles in each of seven slice locations including short-axis and long-axis views may be acquired in approximately 14 seconds at peak stress. Other data acquisition methods may be used such as improved array coils to accelerate scanning, scanning more slices, or scanning each slice for more heartbeats, or using segmented k-space acquisition methods to improve temporal resolution even further.

In an example embodiment of the present invention, immediately following the acquisition of cardiac function images, first-pass cardiac perfusion images are obtained during intravenous infusion of a contrast agent of 0.1 mmol/kg gadolinium-DTPA at a rate of 4 mUs. Other doses or rates may be used. A gradient-echo echo-planar (GRE-EPI) imaging sequence with TR/TE of 5.8/1.2 msec and TSENSE acceleration rate of 2 is used to acquire three short-axis slices each cardiac cycle. A saturation recovery time of 30 msec may be used and an acquisition time per slice of 70 msec (96×160 matrix, 3.0 mm×2.4 mm×10 mm resolution). These sequence parameters appear to be optimal, but there are many more options that are feasible.

Other imaging options that may be used in conjunction with the present invention include: cine only covering more slices and views; perfusion only; cine followed by perfusion; perfusion followed by cine; real-time blood flow velocity mapping; real-time myocardial velocity mapping; real-time cardiac tagging for myocardial strain measurement; real-time displacement encoded stimulated echo (DENSE) for myocardial strain measurement; MR spectroscopy measurement of myocardial metabolism at peak stress; and MR spectroscopy measurement of skeletal muscle metabolism at peak stress.

Following imaging at peak stress, the patient table is removed from the bore of the MR imager 112 in step 812 and diagnostic 12-lead ECG and blood pressure monitoring is performed during the supine recovery period lasting approximately 6-10 minutes. Following this recovery period, the patient is moved again into the bore of the MR imager 112 for additional imaging. Resting cardiac function images and resting first-pass perfusion images are acquired in step 816 using the methods previously described. After another ten minutes to allow the contrast agent to reach equilibrium, delayed myocardial enhancement (DME) (step 818) images are acquired to detect any regions of myocardial infarction or fibrosis. Additional scans may be performed to evaluate valve function, diastolic dysfunction, atrial function, size and compliance of the aorta, and a variety of other common cardiovascular MRI techniques.

During the test, a supervising cardiologist may review interim findings, particularly if they warrant termination of exercise such as severe ischemic ECG changes accompanied by worrisome symptoms. Upon completion of the test, the supervising cardiologist assimilates all of the information including the patient's history, any symptoms recorded during exercise, ECG tracings recorded before/during/after exercise, and the CMR images. Software that displays all the images in a format suitable for rapid review and comparison is used. A comprehensive interpretation of the test results may include assessment of the patients exercise capacity, symptoms and their time of onset as well as mode of resolution, ECG changes, and stress-induced contractile and perfusion response. In addition, CMR allows direct visualization of scarred myocardium that can be incorporated into both segmental and patient-level interpretations of normal/no ischemia, fixed infarction, or ischemic response to stress.

A detailed list of features of the present invention and related advantages are summarized in Table 3.

TABLE 3
Features and Advantages of Invention

| Feature | Advantages |
|---|---|
| Water-based Hydraulic Drive System for | water is safe hydraulic fluid with no risk of combustion |
| Treadmill | or harmful spillage |
| | tap water in plentiful supply |
| | no need to store hydraulic fluids |
| | allows MRI-compatible treadmill to feel similar to |
| | standard treadmills |
| | simple and accessible design allows for easy |
| | maintenance |
| Water-based Hydraulic Elevation System | allows treadmill to safely incline within MRI suite |
| for Treadmill | water is safe hydraulic fluid with no risk of combustion |
| | or harmful spillage |
| | tap water in plentiful supply |
| | no need to store hydraulic fluids |
| | allows MRI-compatible treadmill to feel similar to |
| | standard treadmills |
| Treadmill Control System | allows treadmill run independently through standard |
| | PC as well as with leading treadmill stress testing |
| | software |
| MR Compatibility and Safety | Allows treadmill to be used safely within MRI imaging |
| | suite with no effect on image quality |
| Lift system for treadmill to facilitate | allows treadmill to be raised or lowered to allow easy |
| transfers | transfer of patient from treadmill to MRI table |
| ECG system that is compatible - PC | allows exercise stress test to be performed adjacent |
| outside scan room | to MRI unit |
| Vacuum bags for rapid and accurate | allows rapid acquisition of stress cardiac MR images |
| patient repositioning | that match resting acquisition planes |
| | immediate repositioning enables use of previously |
| | defined image planes, saving considerable time |
| | between exercise and imaging |
| Staff and equipment in same room | eliminates need to transfer patient, personnel, and |
| | monitoring equipment from one room to another |
| | feasible for routine clinical use |
| | replicates stress-echo lab |
| | reduces number of staff required to safely execute |
| | stress study |
| | provides maximum patient privacy |
| Imaging software to optimize at high heart | allows imaging of patients immediately post-stress at |
| rate - heavy breathing | peak heart rates without need for breath holding |

Currently, nearly 10 million cardiovascular stress imaging studies performed annually using echocardiography and nuclear scintigraphy. The present invention allows the superior imaging provided by MR imaging to be used for cardiovascular stress imaging studies. The MR-compatible treadmill system of the present invention supports the use of MR imaging which provides a diagnostic advantage over current echocardiography and nuclear scintigraphy. The present invention allows rapid acquisition of MR images following exercise to more accurately diagnosis cardiovascular disease while increasing patient safety by minimizing the travel required between exercise equipment and the MR imager table.

While certain embodiment(s) of the present invention have been described in detail above, the scope of the invention is not to be considered limited by such disclosure, and modifications are possible without departing from the spirit of the invention as evidenced by the following claims:

1-25. (canceled)

26. A treadmill system for use with an MR imager located in a scan room, comprising:
a treadmill located in the scan room, the treadmill having a belt driven by a motor that produces rotational motion from pressurized fluid; and
a pump located outside the scan room, the pump producing pressurized fluid and being operatively connected to the motor.

27. The system of claim 26, wherein the fluid is hydraulic fluid.

28. The system of claim 27, wherein the fluid is water-based.

29. The system of claim 26, wherein the treadmill is entirely made of non-ferromagnetic materials.

30. The system of claim 26, wherein an equipment room is located outside the scan room and the pump is located in the equipment room.

31. The system of claim 26, wherein the treadmill includes a lift cylinder that varies the gradient of the treadmill belt.

32. The system of claim 31 wherein the lift cylinder operates using pressurized fluid.

33. The system of claim 26, wherein the treadmill further includes a motor brake that limits the speed of the treadmill by constricting the flow of fluid and thereby subjecting the motor to back pressure.

34. A method of conducting an MR study on a patient in a scan room that contains an MR imager and a treadmill that is adjacent the MR imager, that has a belt, and that has a motor that drives the belt by producing rotational motion from pressurized fluid supplied to the motor, comprising the following steps:
pressurizing fluid outside the scan room;
causing the patient to stand on the treadmill belt;
supplying the pressurized fluid to the motor to drive the belt, thereby exercising the patient on the treadmill inside the scan room;
ceasing to supply pressurized fluid to the motor and thereby ceasing to exercise the patient;
transferring the patient from the treadmill to the MR imager without leaving the scan room; and
using the MR imager to conduct an imaging study on the patient.

35. The method of claim 34, wherein said using step comprises the step of conducting an imaging study on the patient's heart.

36. A method of conducting an MR study on a patient in a scan room that contains an MR imager and a treadmill that is adjacent the MR imager, that has a belt, that has a motor that drives the belt by producing rotational motion from pressurized fluid supplied to the motor, and that has a lift cylinder that varies the gradient of the treadmill belt, comprising the following steps:
causing the patient to stand on the treadmill belt;
pressurizing fluid outside the scan room;
supplying the pressurized fluid to the motor to drive the belt;
driving the belt and using the lift cylinder to vary the gradient of the treadmill belt in such a manner as to exercise the patient in accordance with the Bruce Treadmill Exercise protocol on the treadmill inside the scan room;
ceasing to supply pressurized fluid to the motor and thereby ceasing to exercise the patient;
transferring the patient from the treadmill to the MR imager without leaving the scan room; and
using the MR imager to conduct an imaging study on the patient's heart.

37. A method of conducting a cardiac exercise stress test using MR imaging on a patient in a scan room that contains an MR imager and a treadmill that is adjacent the MR imager, that has a belt, and that has a motor that drives the belt by producing rotational motion from pressurized fluid supplied to the motor, comprising the following steps:
pressurizing fluid outside the scan room;
causing the patient to stand on the belt;
supplying the pressurized fluid to the motor to drive the belt, thereby exercising the patient on the treadmill inside the scan room;
continuing to exercise the patient to peak effort;
ceasing to supply pressurized fluid to the motor and thereby ceasing to exercise the patient;
transferring the patient from the treadmill to the MR imager without leaving the scan room;
using the MR imager to conduct an imaging study on the patient's heart;
allowing the patient to recover from the exercise; and
using the MR imager to conduct an imaging study on the patient's heart when the patient's heart is at rest.

38. A method of conducting an MR study on a patient in a scan room that contains an MR imager and a treadmill that is adjacent the MR imager, comprising:
transferring the patient from the treadmill to the MR imager without leaving the scan room.
